(19) United States
(12) Patent Application Publication
(10) Pub. No.: US 2004/0099809 A1
(43) Pub. Date: May 27, 2004

(54) Image intensifying visual scanning device and method

(76) Inventors: Gregoire Verrier, Iselin, NJ (US); Nicolas Vezard, Metuchen, NJ (US)

Correspondence Address: ANTHONY H. HANDAL, KIRKPATRICK & LOCKHART, LLP, 599 LEXINGTON AVENUE, 31ST FLOOR, NEW YORK, NY, 10022-6030, US

(21) Appl. No.: 10/630,621

(22) Filed: Jul. 30, 2003

Related U.S. Application Data
(63) Continuation-in-part of application No. 10/209,439, filed on Jul. 31, 2002.
(63) Continuation-in-part of application No. 09/823,297, filed on Mar. 30, 2001, now Pat. No. 6,636,701.
Application No. 09/823,297 is a continuation-in-part of application No. 09/328,111, filed on Jun. 8, 1999, now Pat. No. 6,262,333.

(51) Int. Cl. G01J 5/00

(52) U.S. Cl. 250/372

(57) ABSTRACT

An electrically powered image intensifier having an output and an input is optically coupled to a zoom lens to receive the focused light image from the lens and provide at the output of the image intensifier an intensified light image. A chassis is configured to support the intensifier and to mainly engage the lens by engaging the mounting structure. The lens is engaged by the mounting structure in a use position, and may be supported by a tripod. The use of the zoom lens allows convenient use in cramped quarters at a short focal length setting, i.e. at a wide angle setting. The intensifier may be used at a distance at a long focal length setting, i.e. at a telephoto setting, advantageously with a tripod and a standard panning handle.

### CROSS REFERENCE TO RELATED APPLICATIONS

This application is a continuation-in-part (“CIP”) of commonly owned U.S. patent application Ser. No. 10/209,439 to Vezard and Verrier, filed Jul. 31, 2002; and a CIP of commonly owned U.S. patent application Ser. No. 09/823,297 to Vezard and Verrier, filed Apr. 30, 2001, which is a CIP of commonly owned U.S. patent application Ser. No. 09/328,811 to Vezard, filed Jun. 9, 1999, now U.S. Pat. No. 6,392,238, dated May 21, 2002.

### STATEMENT REGARDING FEDERALLY SPONSORED RESEARCH OR DEVELOPMENT

Not Applicable.

### FIELD OF THE INVENTION

The present invention is directed to a method and apparatus for the scanning of a crime scene to facilitate the detection of fingerprints and other forensic evidence and employing an image intensifier. More particularly, the present invention is directed to a light imaging system with a selectable field of view, allowing the photographing of such evidence and providing for the viewing of fingerprints and other evidence.

### BACKGROUND OF THE INVENTION

Special tools are frequently used by law enforcement personnel when evaluating a crime scene to collect forensic evidence that may be hard to see or invisible to the human eye. Examples of such evidence include bodily fluids, fingerprints on porous and non-porous surfaces, forged documents, explosive residue, and trace evidence e.g., hair, fibers, etc.

One commonly used tool is a forensic light source that utilizes fluorescent light to detect and record forensic evidence. Subjects illuminated by a forensic light source may be viewed through light filtering goggles, and the output of the source may be filtered to achieve a range of diverse functionalities and corresponding capabilities, with and without the use of chemical developers, powders and dyes.

A recently introduced tool complementary to the forensic light source is a tool that relies on intensified short-wave ultraviolet ( or “uv”) reflectance instead of fluorescence. Such an ultraviolet light tool can reveal otherwise undetectable or difficult to detect evidence on non-porous surfaces where a forensic light source is not useable or the subject requires processing with powders or fluorescent dye. Such systems can increase the area that can be efficiently inspected at a crime scene.

In some conventional forensic tools that utilize ultraviolet light reflectance from an ultraviolet light source a traditional photograph is taken of the output of an intensifier tube. However, photography at the output of an intensifier is limited in resolution by the intensifier.

One ultraviolet light tool that does not rely on the output of an intensifier to obtain a photograph is the “SceneScope” (trademark) ultraviolet light imager or forensic viewer, which is a system with a modified Nikon FM2 camera. This product is available from JY Horiba, Inc. The Scenescope utilizes an intensifier and a modified 35 mm camera to provide the capabilities of both non-intensified photography of reflectance of fingerprints on various backgrounds, and intensified image focusing and viewing in a single instrument. When viewed directly, the output of the intensifier is useful to obtain a focus when using a light source which is not visible to the human eye, such as ultraviolet light (200-400 nm) or infrared light (above 700 nm). In addition, ultraviolet light or infrared light images may be harmful to the naked eye. The intensifier operates electronically to increase the luminescence of the image and provide a visible wavelength output.

In a device of this type, as disclosed in U.S. Pat. No. 6,392,238 (the disclosure of which is incorporated by reference), an ultraviolet forensic viewer comprises a camera having a focusable camera lens to input ultraviolet light images to the camera, the camera is loadable with a film to capture images received from the camera lens. The spacing of the camera lens from the film, when loaded, is adjustable to focus an input ultraviolet image on the film. A camera mirror has an ultraviolet-reflective surface to reflect and image light received from the camera lens to provide a reflected ultraviolet image for use in focusing the camera lens. An image intensifier is optically coupled to the camera to receive the reflected ultraviolet image from the camera mirror and provide a viewable output. The reflected ultraviolet image is focused to the viewable output of the intensifier.

In this device, the camera lens incorporates optical elements which are ultraviolet-transmissive, optionally quartz, and one may employ an ultraviolet filter with the camera lens. The camera (optionally a single lens reflex camera) may comprise a viewing lens system to receive an ultraviolet image reflected by the camera mirror and project that ultraviolet image to the image intensifier. Optionally, an eyepiece or a video camera may be coupled to the intensifier.

### SUMMARY OF THE INVENTION

In accordance with the invention, it has been discovered that, in certain situations, human error is more likely to compromise evidence recognition and/or analysis. In particular, under certain circumstances, for example, in the examination of evidence in relatively cramped quarters, such as, a narrow kitchenette as might be found in a city apartment, or in less accommodating circumstances, such as, the inspection of the inside of an air-conditioning duct, it may be difficult to view a large enough area to effectively scan a crime scene in a time efficient manner. Such panoramic examination of an area may be an important component during an investigation. Such situations compare with normal circumstances where, in a room, for example, one may step back and view a desired larger viewing area.

At the other end of the spectrum, that is in the case of close-up inspection of an area, there are situations where detailed visual analysis of a small area may not be time efficient, practical, or even possible, with a conventional forensic viewer. Consider, for example, inspection of the inside bottom surface of a wastepaper basket. In such circumstances, due to the fact that the officer performing the forensic evaluation of the same cannot introduce his head together with a forensic viewing device into the confined space on the inside of the wastebasket, he is able to perform only a general examination of the entire area at a relatively long distance.

In still other circumstances, close-up or panoramic examination may be possible but may not be optimum because of a variety of human factors. For example, contortion of the body into a difficult position may result in discomfort, pain, distraction, or other mental overhead which may compromise results. In other circumstances, repeated placement of the body in certain non-ergonomic positions in order to accomplish the desired forensic examination may result in cumulative and potentially serious injury. For example, examination of a crime scene in the classic stooped over position of the detective will tend, over time, to cause malformations of the gelatinous cartilage in the lordosis which supports and functions to absorb shock between the vertebrae. In severe cases, this may result in substantial loss of the lordosis and its shock absorbing characteristics, as well as rupture and leakage of the cartilage. In extreme cases, fusing of vertebrae may be required to address the problem.

Given the above difficulties, persons using forensic viewing devices often may compromise by viewing too small or to large an area than they would otherwise deem to be ideal. When the area is too small, additional time is required in order to cover the area. When the area is too large, visual distractions and the relatively small sites to be detected will combine to compromise the quality of the crime scene inspection process.

In accordance with the invention, the above problems are addressed, with substantial effect, through the provision of a forensic viewing device which incorporates, in addition to conventional structures such as filters, photographic film carrying assemblies, image intensifiers and the like, a variable focal length lens to provide a variable field of view. The system may be applied both to single lens reflex systems which allow either photography without intensification or with intensification, as well as systems providing intensified viewing for the human eye. The inventive system may also be applied to forensic viewers which provide an intensified image to a film camera or solid-state video detector array.

In accordance with the invention, a forensic viewer comprises a lens configured to focus light, from an object or surface being imaged, as a focused light image. The lens comprises mounting structure for mounting on the inventive viewer.

More particularly, in accordance with the invention a forensic viewer comprises a lens. The lens comprises a variable focal length configured to focus light in a focal surface, from an object or surface being imaged, as a focused light image of variable image size. The lens has a first operator for varying focal length and image size. The lens comprises a plurality of optical elements. The elements have the characteristics of transmitting ultraviolet light. An imaging device converts an ultraviolet image to a visible image. The imaging device has an input and an output, and support structure for supporting said imaging device at a position relative to the lens where the lens focuses the focused light image at the input of the imaging device. In the inventive forensic viewer 1, the imaging device comprises an electrically powered image intensifier having an output and an input optically coupled to the lens to receive the focused light image from the lens and provide at the output of the image intensifier an intensified light image. Mounting structure is disposed on the lens. A chassis is configured to support the intensifier and to matingly engage the lens by engaging said mounting structure. The lens is engaged by the mounting structure in a use position. An electrical switch has an open and a closed state and is connected to couple electrical power from the power source to the image intensifier in the closed state and to decouple electrical power from the power source to the image intensifier in the open state. The electrical switch comprises an operator for configuring the electrical switch in the open state in an open operator position or in a closed state in a closed operator position. The operator is urged into the closed operator position by the lens mounted on the chassis in the use position.

The lens is moveable, engaged by the mounting structure, between a use position and a release position to release the mounting structure and the lens in the release position.

### BRIEF DESCRIPTION OF THE DRAWINGS

Several embodiments of the invention will now be described by way of example with reference to the accompanying drawings in which:

FIG. 1 is a perspective view of one embodiment of a forensic viewer constructed in accordance with the present invention and suitable for implementation of the method of the present invention;

FIG. 2 is an unexploded perspective view showing the principal parts of the inventive forensic viewer illustrated in FIG. 1;

FIG. 3 is a detail illustrating the operation of a protective switch for the viewer illustrated in FIG. 1;

FIG. 4 is a detail, similar to FIG. 3, showing the switch in the power applying position;

FIG. 5 is a perspective view of the inventive viewer illustrated in FIG. 1 illustrating the front of the system; and

FIG. 6 is a side plan view of the viewer illustrated in FIG. 1 illustrating use with a camera;

FIG. 7 illustrates holding of the inventive viewer of FIG. 1;

FIG. 8 illustrates an alternative embodiment of the inventive viewer;

FIG. 9 illustrates another alternative embodiment of the inventive viewer;

FIG. 10 is a side plan view of an alternative viewer constructed in accordance with the present invention;

FIG. 11 is a schematic diagram of the FIG. 10 viewer; and

FIG. 12 illustrates yet another alternative embodiment of the inventive viewer.

### DETAILED DESCRIPTION

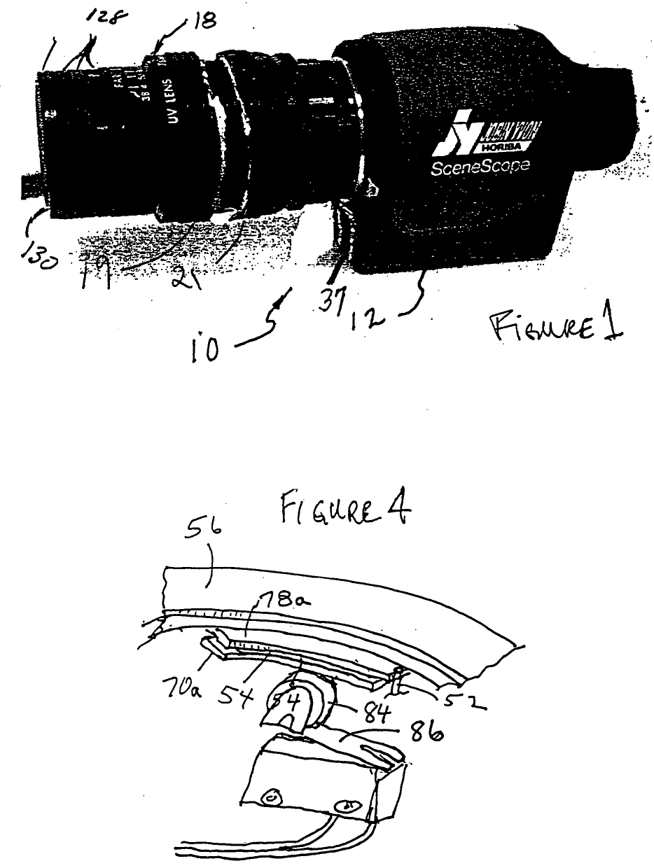

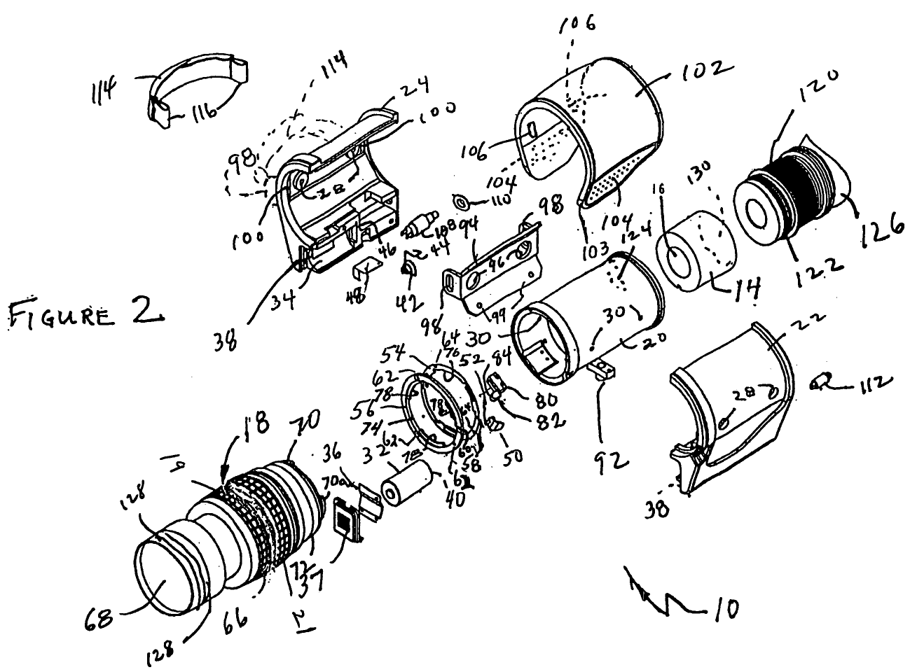

As illustrated in FIG. 1, the inventive forensic viewer 10 comprises a housing 12 which contains an image intensifier 14, as can be seen in the exploded perspective view of FIG. 2. Image intensifier 14 has an input face 16 upon which the image of an object being viewed is imaged. The image of the object being viewed is imaged by a 35 mm camera type lens 18. Lens 18 is of the type commonly referred to as a “zoom lens” and includes an annular operator or cylindrical grip 19 used to focus the lens and a second annular operator or cylindrical grip 21 used to vary the size of the image (and thus the field of view) produced by lens 18. This is done by varying the focal length of lens 18.

Alternatively, lens 18 may have a fixed focal length and zooming provided electronically, for example with a solid state image detector. If desired the ultraviolet sensitivity of a solid state image detecting array may be enhanced by coating with, for example, a material that fluoresces in the visible in the presence of ultraviolet light. In principle, the sensitive surface of a solid-state image detector may be put into contact with the output face of the image intensifier. In accordance with still yet another alternative, the output face of the image intensifier may be separated from the sensitive surface of the solid-state image detector and light output by the image intensifier focused by a lens positioned between the output face and the sensitive surface.

Referring back to the embodiment illustrated in FIG. 1, image intensifier 14 is rigidly supported within a chassis 20. Chassis 20 is rigidly supported within a housing 12. Housing 12 comprises two mating housing members 22 and 24. Mating housing members 22 and 24 are secured by screws (not illustrated) to chassis 20. These screws pass through holes 28 and anchor into tapped holes 30 in chassis 20.

Power to the unit is provided by a battery 32 which is contained within a battery receiving compartment 34 defined by mating half cavities within housing members 22 and 24. Battery 32 is coupled to the circuit by a positive terminal coupler 36 secured to a battery compartment cover 37 which is slidably held in mating recesses 38 in housing members 22 and 24. The negative terminal 40 of battery 32 is coupled to negative terminal coupler 42 which includes a loop 44 which extends around and engages wall 46 on housing member 24.

Plate 48 is made of metal and slides into positive terminal housing member 24. Plate 48 comes into electrical contact with coupler 36, when the battery compartment cover 37 is slidably held in mating recesses 38 in housing members 22 and 24. Plate 48 also has a small hole in which an electrical wire can be soldered. This wire is then connected to interlock switch 80, which controls the removal of power from the image intensifier. Plate 48 closes the electrical circuit with coupler 36, as no wire can be soldered to coupler 36, because the battery compartment cover 37 is removed to replace the battery, and it is not desirable to have any component or wire attached to it.

A key 50 is secured to chassis 20 and has a registration rod 52 for guiding the installation of springy friction member 54 and mounting ring 56 through the use of a pair of holes 58 and 60 respectively. In particular, registration rod 52 passes through holes 58 and 60 thus ensuring that they are maintained in proper relationship to each other and the rest of the system. Springy friction member 54 and mounting ring 56 are held in place by three substantially equispaced screws (not illustrated), two of which pass through holes 62 and 64 in mounting ring 56 and springy friction member 54, respectively.

Lens 18 a number of filtering optical members 68. Lens 18 is provided with a so-called bayonet mounting comprising a number of circumferential studs 70, which extend outwardly and radially from the base 72 of the body of lens 18. Studs 70 are positioned radially to be able to pass through the inner side walls 74 and 76 of mounting ring 56 and springy friction member 54, respectively, between inwardly extending mating studs 78. Studs 70 mate with mating studs 78. More particularly, after insertion of lens 18, while it is in the angular position illustrated in FIG. 2, lens 18 is rotated clockwise into the mating position where studs 70 have their outwardly facing surfaces bearing against that portion of the outwardly facing surface of springy friction member 54 which overlies the inwardly facing surfaces of circumferential studs 70.

During installation of lens 18 onto viewer 10, rotation of lens 18 beyond the mating position is prevented by registration rod 52.

Alternatively, other lens mounting structures may be used such as screw or snap mounts.

Power is coupled from battery 32 to image intensifier 14 by a single-pole single-throw switch 80 which is capable of assuming either a closed or open state. The state of switch 80 is controlled by operator 82 which is a lever which is pivotally mounted at one end and is terminated at the other end in a plastic cam follower 84. As illustrated most clearly in FIG. 3, when lens 18 is not mounted in viewer 10, cam follower 84 bears against that portion of inward surface of springy friction member 54, which faces its respective stud 78.

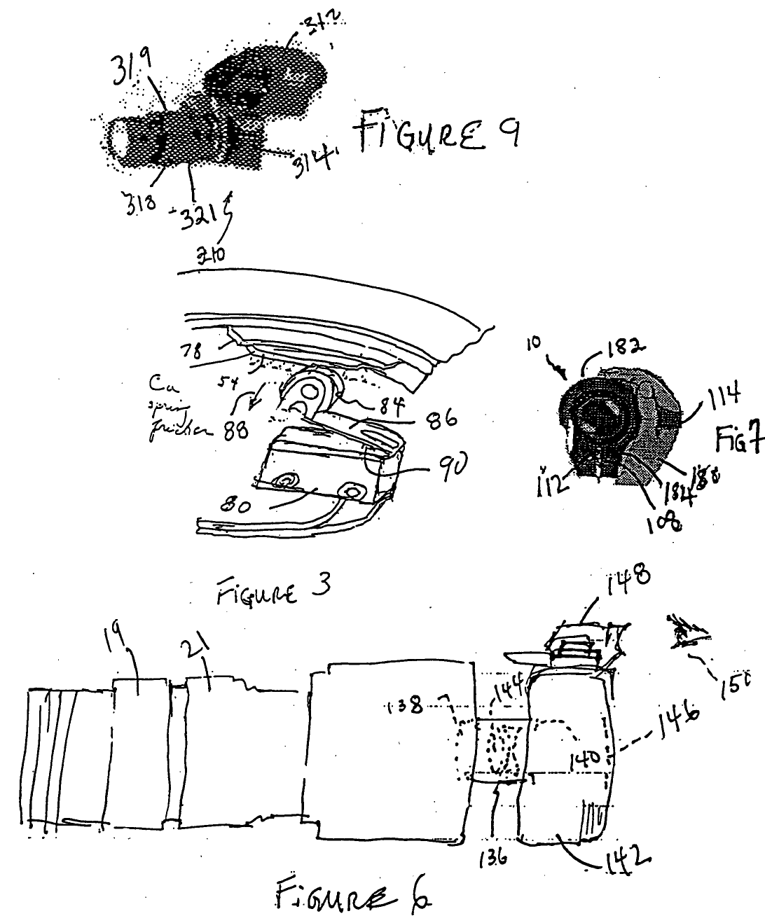

When lens 18 is inserted into the bayonet mounting formed by studs 78 on mounting ring 56, and rotated, studs 70 pass underneath studs 78. In the case of studs 70a and 78a, stud 70a passes between cam follower 84 and that portion of springy friction member 54 which overlies stud 78a, resulting in advancing lever arm 86 in the direction of arrow 88, moving arm 86 from the position illustrated in FIG. 3 to the position illustrated in FIG. 4. This results in the depression of switch button 90, causing switch 80 to assume the closed state and conduct electricity to image intensifier 14, thus powering the image intensifier and allowing the system to operate.

Conversely, when lens 18 is rotated in the counterclockwise direction, stud 70a is also rotated in the counterclockwise direction pulling it out, from in between cam follower 84 and stud 78a, causing lever arm 86 to spring back from the position illustrated in FIG. 4 to the position illustrated in FIG. 3.

Support plate 92 is inserted into and maintained in position when housing members 22 and 24 are assembled together. Additionally, two screws (not illustrated) maintain support plate 92 in position in viewer 12. Support plate 92 is tapped with a ¼×20 thread. This thread size is standard on any photographic camera. Support plate 92 is used to fix the viewer on any tripod or copy-stand when a photo must be taken and mates with the tripod camera securing bolt. The use of the zoom lens allows convenient use in cramped quarters at a short focal length setting, i.e. at a wide angle setting. The intensifier may be used at a relatively long distance at a long focal length setting, i.e. at a telephoto setting, advantageously with a tripod and a standard panning mechanism and handle. Likewise, panning may be motorized or computer controlled.

Bracket 94 includes a pair of holes 96 through which the screws, which secure mating housing member 24 to chassis 20, pass. Bracket 94 also includes a pair of strap engaging loops 98 which pass through holes 100 in mating housing member 24. This allows them to extend out of the housing when the inventive viewer 10 is assembled in the manner illustrated in dashed lines in FIG. 2. Bracket 94 also includes a pair of holes 99, through which screws, which secure bracket 94 to chassis 20, pass.

A rubbery soft comfort cowl 102 overlies a relatively stiff plastic cover 103. Cowl 102 includes gripping surfaces 104 on both its sides, and also bears against a pair of elongated holes 106 through which strap engaging loops 98 extend when the inventive viewer 10 is fully assembled.

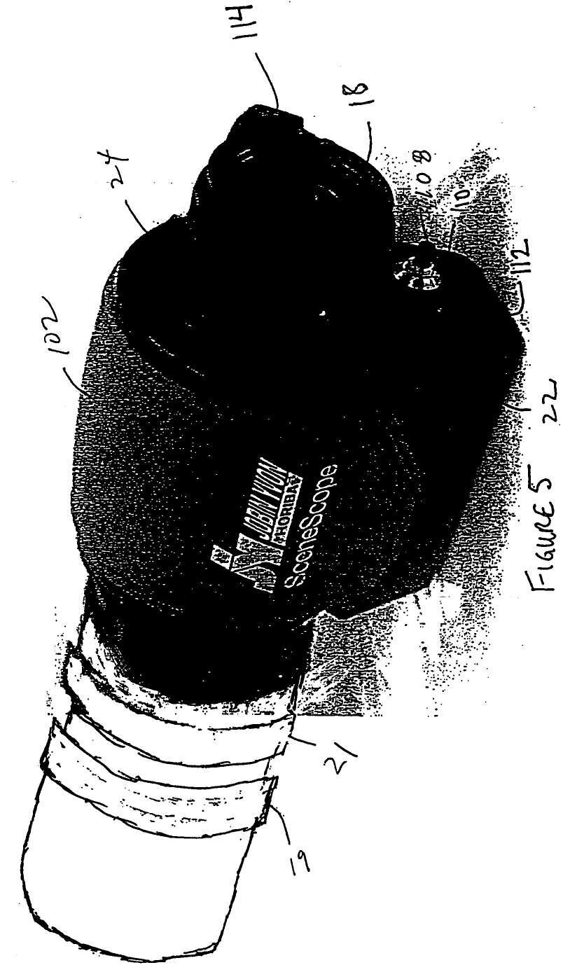

As is illustrated in FIG. 5, an on off switch 108 is secured in position by a nut 110. On-off switch 108 is a push button switch which is changed between the open and closed state by repeated depression of its associated push button which extends through mating housing member 24. In similar fashion, mating housing member 22 has a pilot light 112 mounted in it.

A strap 114, which includes a pair of fabric strap loops 116, completes the structure. Fabric strap loops 116 engage strap engaging loops 98 which may be made of metal, in contrast to most of the other structural parts which may be made of plastic. If desirable, threaded parts and chassis 20 may be made of metal. It is noted that the top surface 118 of cowl 102 is rounded to accommodate the palms and inside surfaces of the fingers of the hand of an operator of the inventive viewer 10, who has passed his hand through the strap 114 in the fashion of a video camcorder.

Viewing by the human eye is accommodated by an eyepiece 120, which includes threads 122 which engage mating threads 124 on the inside of chassis 20. The eyepiece 120 also includes a rubbery eyecup 126 to exclude ambient light while at the same time providing comfort to the user.

In accordance with the preferred embodiment of the invention, it is contemplated that one or more filters 128 may be attached to lens 18. The system will also accommodate a lens cap 130 which performs the function of protecting the optics of the system.

When it is desired to use the inventive system when illuminating a subject with ultraviolet light, an ultraviolet filter, such as filter 128 may be mounted on lens 18 to eliminate all but ultraviolet light from entering the system. Of course, if light produced by other physical effects are being detected, other filters, such as bandpass filters tuned to an expected wavelength range of emissions, may be employed on lens 18.

During use, light admitted into lens 18 is focused on the front face 16 of image intensifier 14 resulting in the production of a bright image on the output face 130 of the image intensifier. This image is focused by eyepiece 120 for viewing by the human eye.

As has been alluded to above, it is contemplated that in some, but not all cases, the inventive viewer 10 will be used in connection with the observation of ultraviolet light images. Accordingly, it is desirable that lens 18 have the characteristic of efficiently transmitting ultraviolet light, and that the lens is made of suitable materials, such as quartz, and/or lenses including appropriate coatings, where operation in conjunction with ultraviolet images is desired.

In the event that lens 18 is removed from the inventive viewer 10, the rotation of lens 18 necessary for removal results in moving switch 80 from the position illustrated FIG. 4 to the position illustrated FIG. 3, resulting in the removal of power from image intensifier 14. As a result, image intensifier 14 is not subjected to what are likely to be excessive light intensities while under power. Accordingly, with switch 80 in the open position, there is no power applied to image intensifier 14, and image intensifier 14 can therefore sustain exposure to light without damage.

If desired, a standard 35 mm SLR camera with a standard photographic zoom lens may be connected directly behind eyepiece 120. The same way a user of such a camera sees the intensified image with his eye through the eyepiece 120, a 35 mm single lens reflex (SLR) camera, equipped with its photographic zoom lens (its eye), will see through the eyepiece the same intensified image. See the description of FIG. 8 below.

If desired, an adapter which screws into chassis 20 in place of eyepiece 120 may be used to attach to a photographic film camera for permanently recording images produced by the inventive viewer 10. Referring to FIG. 6?, such an adapter 136 has threads 138 that mate with the inside threads in chassis 20. At the other end, adapter 136 has mounting structure 140 similar to studs 70 which permit the inventive viewer 10 to be attached to an ordinary 35 mm camera 142 in much the same manner as a conventional 35 mm lens. Adapter 136 also includes optics 144 for focusing the image from image intensifier 14 onto film 146 in the body of 35 mm camera 142 which has been equipped with the inventive viewer, as if the inventive viewer where a specialized lens or other accessory.

Viewfinder optics 148 of the type customarily used in a single lens reflex camera reflect the focused light image from the output of the image intensifier for viewing of the intensified image on the output of the image intensifier by a user 150.

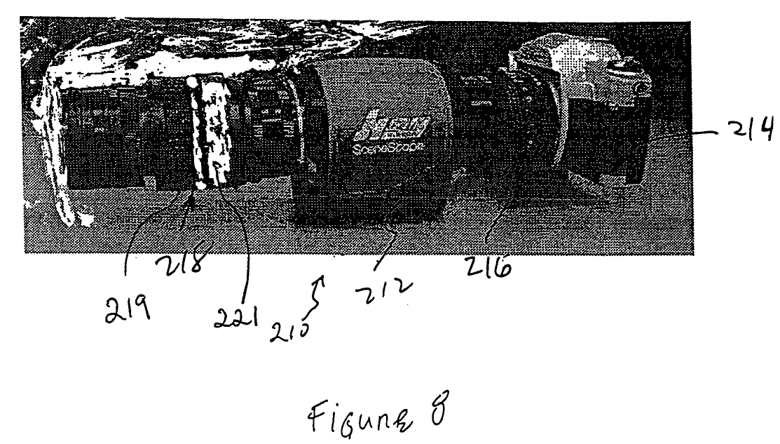

As shown in FIG. 7, the inventive viewer 10 may be comfortably gripped by the hand 180 of a user on account of the round top surface 182 of the same. Comfort and sure grip is promoted by narrow portion 184 and the grippable rubber surface of cowl 102.

In accordance with another embodiment of the invention, as illustrated in FIG. 8, inventive system 210 comprises an intensifier unit like that of the earlier embodiment including the inventive protection switch. A camera 214 with a zoom lens is optically and mechanically coupled to the output of intensifier 212. Zoom lens 218 is coupled to intensifier 212 and when removed opens the protection switch. Zoom lens 218 includes a focusing ring 219 and a zooming ring 221.

In accordance with another embodiment of the invention, as illustrated in FIG. 9, the inventive system 310 comprises an intensifier unit, like that of the earlier embodiment, and including the inventive protection switch. An SLR camera 314 with a modified viewfinder optic 216 provides its viewfinder output to intensifier 312. Intensifier 312 is optically and mechanically coupled to the viewfinder output. A lens 218 is coupled to camera 314. Zoom lens 318 includes a focusing ring 319 and a zooming ring 321. When the intensifier 312 is removed from the viewfinder, coupling structure opens the protection switch situated between the intensifier and the viewfinder optic.

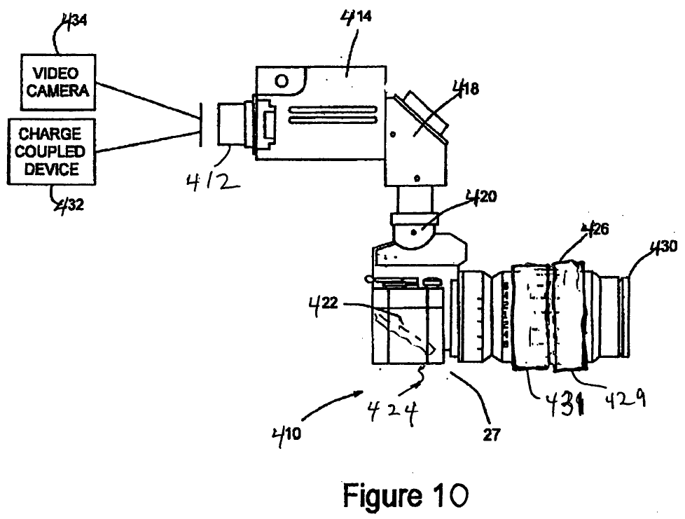

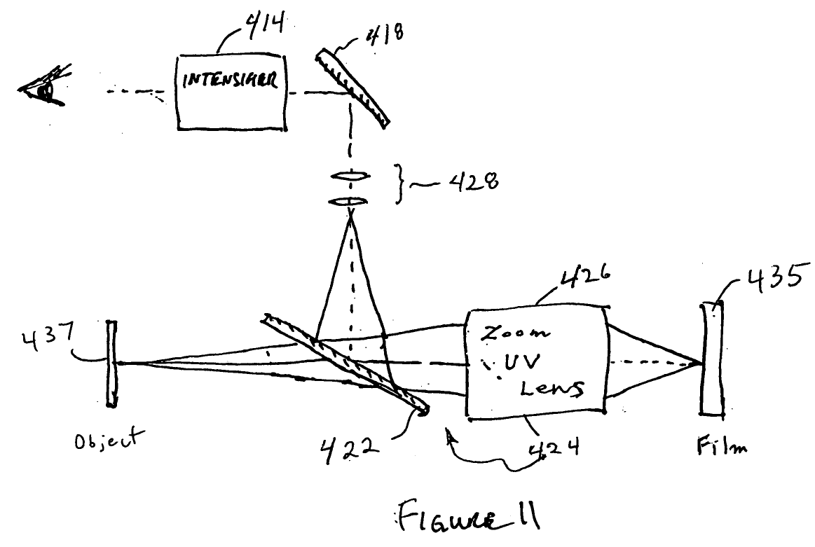

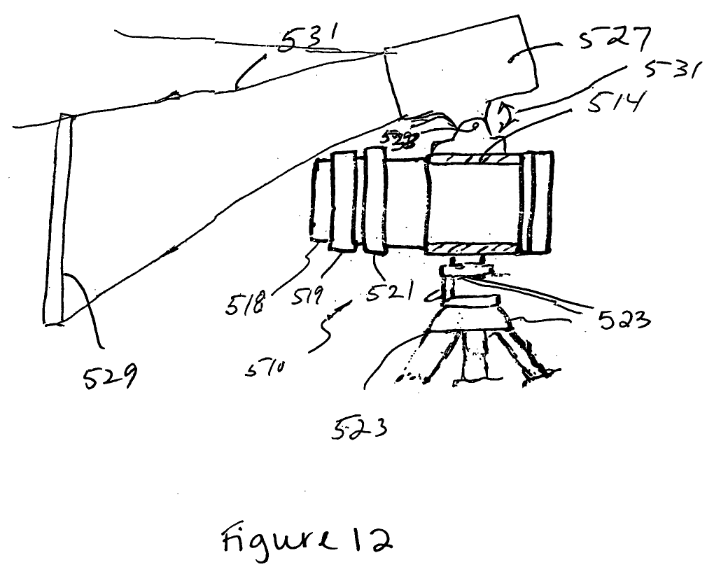

Still another embodiment of the present invention is illustrated in FIGS. 10 and 11. UV-Imager system 410 includes a camera 424. Camera 424 can be a modification of a conventional camera. In one embodiment, camera 424 is a modified FM2 camera from Nikon Corp. The conventional Nikon FM2 camera is modified by removing the mirror included with the camera and replacing it with a UV reflective coated mirror 422. The UV reflection coating of mirror 422 reflects light with wavelengths within the range of approximately 200-3600 nm. In one embodiment, the coating of UV coated reflective mirror 422 provides peak reflection at approximately 254 nm, and reflects 90-95% of UV light compared to 20-25% reflection for a conventional mirror.

Camera 424 further includes a dual lens system 428 (FIG. 11) that replaces the prism found in a conventional camera. In one embodiment, the dual lens system includes two lenses, each of which is a silica lens with a focal length of 50 mm and diameter of 22.5 mm. In addition, camera 424 does not have a focusing screen as is included in conventional cameras. Camera 424 is loaded with a UV sensitive film. In one embodiment, the film is Kodak Tri-X 400 ASA.

Camera 424 includes a UV transmissive camera lens 426. Camera lens 426 can be any known manual or motorized zoom lens that has lens elements made of UV transmissive material. In accordance with a preferred embodiment of the invention, camera lens 426 includes a focusing ring 429 and a zooming ring 431.

Coupled to lens 426 is a 254 nm filter 430. In another embodiment, two stacked 254 nm filters 430 are coupled to lens 426. This embodiment is more suitable for outdoor use.

Camera 424 is coupled to a second mirror holder 418 by a lockable mechanism 420. Lockable mechanism 420 allows second mirror holder 418 to be adjusted relative to camera 424. Second mirror holder 418 includes a UV reflective coated mirror (not shown) having a coating reflecting UV within the range of approximately 200-360 nm. Second mirror holder 418 is coupled to an intensifier 414. Intensifier 414 intensifies light received from second mirror holder 418 so that the light image can be directly viewed or sent to a charge-coupled device. (“CCD”). Intensifier 414 can be a first, second, or third generation intensifier, or merely a UV to green light converter.

An eyepiece 412 is coupled to intensifier 414. Eyepiece 412 can be used for direct eye viewing. Further, a CCD 432 or a video camera 434 can be coupled to eyepiece 412 to provide video monitor viewing/focusing or videotaping while the objects are photographed on the UV film 435. In combination, UV-imager system 410 allows a fingerprints or other forensic object 437 to be photographed by camera 424, or simultaneously viewed through eyepiece 412. The UV-reflective coating on the mirrors of system 410 boosts the UV reflectance which enhances the image of the objects. The removal of the focusing screen and the prism from camera 424 increases the UV transmission towards intensifier 414. Dual lens system 428, between mirror 424 and second mirror holder 418, re-images the objects as a flat field onto the front face of intensifier 414.

As illustrated in FIG. 12, another embodiment of the inventive system 510 may include a zoom lens 518 having a focusing ring 519 and a zooming ring 521. Lens 518 provides light to an image intensifier 514 which, if desired, may be mounted on a tripod 523, which is of particular value when inspecting a crime scene with zoom lens 518 set at a setting with a relatively long focal length corresponding to telescopic magnification of the area being inspected. The steadiness of a tripod is of importance because of the relatively large displacements of the image, produced by the inventive system, caused by relatively minor movements of an unmounted system. In accordance with the present invention, it is contemplated that tripod 523 includes standard tripod mounting mechanisms including a handle 525 which allows panning and other movements with relatively steadiness, thus increasing the quality of the image produced and increasing the speed with which a crime scene can be inspected.

In accordance with this embodiment of the invention, the system further includes a light source 527 which is used to illuminate an object being viewed such as object 529. In particular, light 527 is aimed at the object 529 to illuminate it. In accordance with the preferred embodiment light 527 is an ultraviolet source and lens 518 may include filters to block ultraviolet light (of particular value in the case of using the system in conjunction with film and it is desired to detect light other than ultraviolet light or to pass expected or observed wavelengths of emission. In order to accommodate objects at different distances from system 510, light source 527 is mounted on a swivel mount including a pin 533 for rotatable support of light source 527, allowing movement in the directions indicated by arrow 531.

It is noted that the use of a light source 527 attached to the inventive forensic viewer may be used in connection with any of the embodiments illustrated in FIGS. 1-11, with and without the use of a tripod.

Several embodiments of the present invention are specifically illustrated and/or described herein. However, it will be appreciated that modification and variations of the present invention are covered by the above teachings and within the purview of the appended claims without departing from the spirit and intended scope of the invention.

1. A forensic viewer, comprising a lens, said lens comprising a variable focal length lens configured to focus light in a focal surface, from an object or surface being imaged, as a focused light image of variable image size, said lens having a first operator for varying focal length and image size, said lens comprising a plurality of optical elements, said elements having the characteristics of transmitting ultraviolet light, an imaging device for converting an ultraviolet image to a visible image, said imaging device having an input and an output, and support structure for supporting said imaging device at a position relative to said lens where said lens focuses said focused light image at the input of said imaging device;

2. A forensic viewer as in claim 1, wherein said imaging device comprises an electrically powered image intensifier having an output and an input optically coupled to the lens to receive the focused light image from the lens and provide at the output of said image intensifier an intensified light image.

3. A forensic viewer, as in claim 2, further comprising:
(a) mounting structure disposed on said lens;
(b) a chassis configured to support said intensifier and to matingly engage said lens by engaging said mounting structure, said lens being engaged by said mounting structure in a use position;
(c) a power source; and
(d) an electrical switch having an open and a closed state, connected to couple electrical power from said power source to said image intensifier in said closed state and to decouple electrical power from said power source to said image intensifier in said open state, said electrical switch comprising an operator for configuring said electrical switch in the open state in an open operator position or in a closed state in a closed operator position, and said operator being urged into said closed operator position by said lens mounted on said chassis in said use position.

4. An image intensifying forensic viewer as in claim 3, wherein said lens is moveable, engaged by said mounting structure, between a use position and a release position to release said mounting structure and said lens in the release position.

5. A forensic viewer as in claim 1, wherein said imaging device is an electrically powered image intensifier, said image itensifier being sensitive to ultraviolet light.

6. A viewer as in claim 1, further comprising a UV light source configured to illuminate said object or surface.

7. A viewer as in claim 1, further comprising a tripod mounting, secured to said support structure.

8. A viewer as in claim 1, wherein said imaging device is a layer of material which converts ultraviolet light to visible light.

9. An image intensifying forensic viewer as in claim 3, wherein said lens has a bayonet mounting structure configured to nest into and be rotated into engagement with mating bayonet mounting structure on said chassis.

10. An image intensifying forensic viewer as in claim 3, wherein said lens transmits ultraviolet light, said electronically powered device image intensifier is sensitive to ultraviolet light and said lens is a focusable lens.

11. An image intensifying forensic viewer as in claim 2, wherein said lens has snap mounting structure configured to snap into engagement with mating snap mounting structure on said chassis.

12. An image intensifying forensic viewer as in claim 3, further comprising viewing optics for presenting the output of said image intensifier to the eye of a viewer.

13. An image intensifying forensic viewer as in claim 1, further comprising viewing optics for presenting the output of said image intensifier to the eye of a viewer.

14. An image intensifying forensic viewer as in claim 13, further comprising viewing optics for presenting the output of said image intensifier to photographic film in a camera.

15. An image intensifying forensic viewer as in claim 3, wherein said operator is spring biased in said open operator position.

16. A forensic viewer according to claim 1 further comprising an ultraviolet filter positioned in front of said lens.

17. A forensic viewer according to claim 3 further comprising an ultraviolet filter positioned in front of said lens.

18. A viewer according to claim 3 comprising an eyepiece coupled to said intensifier.

19. An ultraviolet forensic viewer according to claim 17, comprising a camera containing film coupled to receive said focused light image from said film.

20. A forensic viewer as in claim 1, wherein said support structure comprises a housing with a round top and narrowed bottom and further comprising a hand strap coupled at two points to the side of said housing.

21. A forensic viewer as in claim 20, wherein said housing round top is made of a soft grippable material.

22. A forensic viewer as in claim 21, wherein said housing is narrower at the bottom of the housing as compared to the top of the housing.

23. A forensic viewer according to claim 1 further comprising an ultraviolet light source mounted on said support structure.

24. A forensic viewer according to claim 3 further comprising an ultraviolet light source mounted on said support structure.

25. A forensic viewer as in claim 1, wherein said focal length of said lens is varied by an automated system.

26. A forensic viewer as in claim 25, wherein said automated system is an electronic zoom without moving parts.

27. An ultraviolet light imager comprising:
a) a camera having:
i) a variable focal length camera lens to input ultraviolet light images to the camera, the camera being loadable with a film to capture images received from the camera lens, the spacing of the camera lens from the film, when loaded, being adjustable to focus an input ultraviolet image on the film; and
ii) a camera mirror having an ultraviolet—reflective surface to reflect image light received from the camera lens to provide a reflected ultraviolet image for use in focusing the camera lens; and
b) an image intensifier optically coupled to the camera to receive the reflected ultraviolet image from the camera mirror and provide a viewable output;
wherein the reflected ultraviolet image is focused to the viewable output of the intensifier.

28. An ultraviolet light imager according to claim 27 wherein the camera lens is an ultraviolet—transmissive, optionally quartz, lens.

29. An ultravioletlight imager as in claim 28, further comprising a tripod mounting for supporting said imager.

30. An ultraviolet light imager according to claim 27 comprising a second mirror having an ultraviolet—reflective surface, optionally a surface reflecting ultraviolet light at wavelengths from about 200 to about 360 nm, the second mirror being capable of reflecting to the intensifier images received from the camera mirror.

31. An ultraviolet light imager according to claim 27 comprising an eyepiece coupled to the intensifier.

32. An ultraviolet light imager according to claim 31 comprising a charge-coupled device coupled to the eyepiece.

33. An ultraviolet light imager according to claim 32 comprising a video camera coupled to the eyepiece.

34. An ultraviolet light imager according to claim 27 wherein the intensifier is a UV—to—green light converter.

35. An ultraviolet light imager according to claim 27 comprising an ultraviolet—sensitive film loaded in the camera.

36. An ultraviolet light imager comprising:
a) a camera having:
i) a variable focal length camera lens to input ultraviolet light images to the camera, the camera lens being an ultraviolet—transmissive, optionally quartz, lens;
ii) a film to capture images received from the camera lens, the spacing of the camera lens from the film being adjustable to focus an ultraviolet image on the film; and
iii) a camera mirror having an ultraviolet—reflective surface to reflect a portion of the image light received from the camera lens to provide a reflected ultraviolet image for use in focusing the camera lens;
b) an image intensifier optically coupled to the camera to receive the reflected ultraviolet image from the camera mirror and provide a viewable output;
c) a viewing lens system to receive an ultraviolet image reflected by the camera mirror and project said ultraviolet image to the image intensifier, said lens system replacing the conventional prism and focusing plate of a single lens reflex camera; and
d) a second mirror having an ultraviolet—reflective surface, optionally a surface reflecting ultraviolet light at wavelengths from about 200 to about 360 nm, the second mirror being capable of reflecting to the intensifier images received from the camera mirror;
wherein the reflected ultraviolet image is focused to the viewable output of the intensifier.

37. An invisible wavelength light imager comprising:
a) a camera having:
i) a variable focal length camera lens to input invisible wavelength light images to the camera;
ii) a film to capture images received from the camera lens, the spacing of the camera lens from the film being adjustable to focus an invisible wavelength image on the film; and
iii) a camera mirror having an invisible wavelength—reflective surface to reflect a portion of the image light received from the camera lens to provide a reflected invisible wavelength image for use in focusing the camera lens; and
b) an image intensifier optically coupled to the camera to receive the reflected invisible wavelength image from the camera mirror and provide a viewable output;
wherein the reflected invisible wavelength image is focused to the viewable output of the intensifier.

38. A method of examining a crime scene, comprising:
a) illuminating the crime scene with ultraviolet light;
b) using a zoom lens with an adjustable focal length to project an ultraviolet image of a subject on an image intensifier for intensification; and
c) adjusting the focal length of said zoom lens; a method as in claim 19, further comprising:
d) using the intensified ultraviolet image to focus the camera on the subject; and
e) opening the camera shutter to capture a focused ultraviolet image of the subject on film.
